(19) United States
(12) Patent Application Publication
(10) Pub. No.: US 2026/0198954 A1
(43) Pub. Date: Jul. 16, 2026

(54) DEVICES, SYSTEMS, AND METHODS FOR SEPARATING TISSUE WITH A BLUNT DISSECTOR AND A FIXATION MEMBER

(71) Applicant: BOSTON SCIENTIFIC SCIMED, INC., Maple Grove, MN (US)

(72) Inventors: Thomas Neudeck, Austin, TX (US); Stephen West, Austin, TX (US); Vladimir Mitelberg, Austin, TX (US); Timothy Noah Foster-Frau, Austin, TX (US)

(73) Assignee: BOSTON SCIENTIFIC SCIMED, INC., Maple Grove, MN (US)

(21) Appl. No.: 19/439,809

(22) Filed: Jan. 5, 2026

Related U.S. Application Data
(60) Provisional application No. 63/742,104, filed on Jan. 6, 2025.

(51) Int. Cl. A61B 17/32; A61B 17/00; A61B 17/3209

(52) U.S. Cl. CPC A61B 17/320016; A61B 17/3209; A61B 2017/00544; A61B 2017/00557; A61B 2017/320048

(57) ABSTRACT

A tissue separator having a tissue-separating end effector with a blunt dissector, such as an expandable member configured to separate tissue, and a fixation member. The fixation member inhibits and preferably prevents rebound of the expandable member as the expandable member expands against tissue to separate the tissue. The expandable member may be an inflatable balloon or tunnelling balloon. The fixation member may be an expandable member. The fixation member may be fixed in close proximity to the proximal end of the expandable member, or may be movable with respect to the expandable member.

### CROSS-REFERENCE TO RELATED APPLICATIONS

This application claims the benefit of priority under 35 U.S.C. § 119 to U.S. Provisional Application No. 63/742,104, filed Jan. 6, 2025, the entire disclosure of which is hereby incorporated by reference herein for all purposes.

### FIELD

The present disclosure relates generally to the field of medical devices, systems, and methods for separating tissue. More particularly, the present disclosure relates to medical devices, systems, and methods using an expandable member to separate tissue, and an anchoring member to anchor the device with respect to tissue to be separated.

### BACKGROUND

During endoscopic procedures, at times it is necessary to separate tissue layers at a treatment site. For instance, to enable procedures such as endoscopic submucosal dissection (ESD) to be performed, the submucosal layer and the muscularis layer of intestinal tissue may need to be separated. The resulting pocket of tissue may be referred to as third space. Once the endoscope is inserted into this third space, the procedure is sometimes referred to as third space endoscopy. Typically, electrosurgical tools or ESD knives are used to dissect tissue after injection of a lifting agent to raise the lesion. Electrosurgical tools may be difficult to control, which may lead to undesired cutting of tissue and/or blood vessels. Using electrical energy to dissect tissue in the gastrointestinal (GI) tract can have drawbacks or other adverse events, such as, without limitation, injuries to the muscularis layer which can increase the risk for delayed bleeding; perforations; post-ESD electrocoagulation syndrome (PECS); inadvertent dissection of large blood vessels which need to be treated immediately and which can significantly increase procedure time and negatively impact visualization; etc. These challenges can require a lot of training and practice to perform endoluminal tissue dissections safely and effectively, and thereby can limit wider adoption and case volumes.

Blunt tissue separation using blunt instruments (or even the surgeon's finger tip) has been demonstrated to potentially mitigate some of the issues described above. Insertion of a blunt instrument between the muscle and submucosa applies tension to the tissue layers which results in “ensile” tissue separation without using electrical energy/heat. Blunt or mechanical or “cold” tissue separation (without use of heat or electrical energy, such as applied by electrosurgical instruments) might not have some of the limitations described above, and may increase the overall efficiency and safety during an ESD procedure, and may reduce the learning curve for inexperienced users. For instance, the tissue composition of the muscularis layer is different from submucosal tissue, allowing for a clean separation of these tissue layers when sufficient tension is applied by a blunt instrument, without tearing or otherwise disrupting each discrete layer. Additionally, larger blood vessels are more elastic than the connective tissue between the tissue layers, and are less likely to be separated using cold tissue separation (in contrast with inadvertent cutting which may occur with sharper cutting instruments or electrosurgical cutting instruments). The use of blunt tissue dissection is common practice in open and laparoscopic surgery to separate organs and/or tissue layers, and/or to isolate blood vessels. Blunt cauterization may be used prior to, sometime obviating the need for, cauterization using energy. Blunt cauterization often is the main means to separate tissue during open surgery so that blood vessels can be isolated and treated conservatively. However, blunt tools used during endoscopic procedure present various challenges which lead medical professionals to use electrosurgical tools instead.. One issue that has been observed with the use of blunt instruments to separate tissue is that as the blunt instrument is advanced to further separate tissue, the encounter of the blunt instrument with a tissue surface typically causes the blunt instrument to be pushed backward and rebound away from the tissue to be separated and into the pocket where tissue has already been separated. To ensure continued tissue separation, the medical professional has to apply sufficient forward pressure to the blunt instrument at the endoscope biopsy port to advance the blunt instrument into the tissue to separate the tissue as well as to overcome the counterforces from the tissue. This can make endoscope stabilization and maneuvering more challenging, and may negatively affect tissue targeting and dissection precision of the device, system, and method.

As may be appreciated, solutions to these and other challenges in the art would be welcome.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a tissue separator is capable of being navigated transluminally to a treatment site within a patient and insertable through an incision formed in tissue at the treatment site to create a third space within the tissue. In some aspects, the tissue separator includes a flexible elongate member configured to be navigated within a curved or tortuous body passage to the treatment site and having a distal end; and an end effector positioned along the distal end of the flexible elongate member and configured to be inserted through the incision in the tissue at the treatment site, the end effector including an expandable member at the distal end of the flexible elongate member, and a fixation member adjacent and proximal to the expandable member.

In some aspects, the expandable member is an inflatable balloon. Additionally or alternatively, the fixation member is an inflatable ballon. In some aspects, the flexible elongate member defines a first lumen fluidly communicating inflation medium to the expandable member, and a second lumen fluidly communicating inflation medium to the fixation member.

In some aspects, the expandable member is a tunneling balloon.

In some aspects, the fixation member is expandable to a fixation size. In some aspects, the expandable member is expandable to a maximum size smaller than the fixation size of the fixation member. In some aspects, when the fixation member is expanded to the fixation size and the expandable member is expanded to the maximum size thereof, the expandable member and the fixation member are immediately adjacent each other without interfering with movement of each other.

In some aspects, the fixation member is an inflatable balloon.

In some aspects, the fixation member is mounted on a distal end of a sheath axially translatable over the flexible elongate member.

In accordance with various principles of the present disclosure, a system is described for separating tissue within a patient to create a third space within the tissue. In some aspects, the system includes a cutting device capable of being navigated transluminally within a patient's body to form an incision in the tissue; and a tissue separator capable of being navigated transluminally to the treatment site and insertable through an incision formed by the cutting device and into the tissue. In some aspects, the tissue separator includes a flexible elongate member configured to be navigated within curved or tortuous body passages to the treatment site and having a distal end; and an end effector positioned along the distal end of the flexible elongate member and configured to be inserted through the incision formed by the cutting device, the end effector comprising an expandable member at the distal end of the flexible elongate member, and a fixation member proximal to the expandable member.

In some aspects, the expandable member is an inflatable balloon.

In some aspects, the expandable member is a tunneling balloon.

In some aspects, the fixation member is expandable to a fixation size. In some aspects, the expandable member is expandable to a maximum size smaller than the fixation size of the fixation member. In some aspects, when the fixation member is expanded to the fixation size and the expandable member is expanded to the maximum size thereof, the expandable member and the fixation member are immediately adjacent each other without interfering with movement of each other.

In some aspects, the fixation member is mounted on a distal end of a sheath axially translatable over the flexible elongate member.

In some aspects, the fixation member is an inflatable balloon.

In accordance with various principles of the present disclosure, a method of creating third space within tissue within a patient's body includes inserting a distal end of an end effector of a tissue separator through an incision in tissue within a patient's body and into the tissue; expanding an expandable member on the distal end of the end effector within the tissue to separate the tissue and to create an initial third space within the tissue; collapsing the expandable member; expanding a fixation member of the end effector within the initial space within the tissue to anchor the end effector with respect to the tissue; and expanding the expandable member to further separate tissue to expand the size of the initial third space within the tissue.

In some aspects, the method further includes collapsing the expandable member, collapsing the fixation member, advancing the end effector within the tissue, expanding the fixation member, and expanding the expandable member to increase the size of the third space within the tissue.

In some aspects, the expandable member is an inflatable balloon, and expanding the expandable member comprises inflating the expandable member. In some aspects, the fixation member is an inflatable balloon, and expanding the fixation member comprises inflating the fixation member.

In some aspects, expanding the expandable member to further separate tissue comprises expanding the expandable member up to a size at which the expandable member is immediately adjacent the fixation member.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an elevational view of a tissue-separating system formed in accordance with aspects of the present disclosure.

FIG. 2 illustrates a cross-sectional view along line II-II of FIG. 1.

FIG. 3 illustrates an elevational view of another tissue-separating system formed in accordance with various principles of the present disclosure.

FIG. 4 illustrates a cross-sectional view along line IV-IV of FIG. 3.

FIG. 5 illustrates an elevational view of another tissue-separating system formed in accordance with various principles of the present disclosure.

FIG. 6 illustrates a cross-sectional view along line VI-VI of FIG. 5.

FIG. 7 illustrates a schematic representation of a treatment site with a cutting device of a tissue separating system, formed in accordance with various principles of the present disclosure, creating an initial incision.

FIG. 8 illustrates a schematic representation similar to that of FIG. 7, but with the tissue-separating end effector of a tissue separator, formed in accordance with various principles of the present disclosure, being inserted through the incision illustrated in FIG. 7.

FIG. 9 illustrates a schematic representation similar to that of FIG. 8, but with an expandable member of the tissue-separating end effector expanded to effect initial separation of tissue.

FIG. 10 illustrates a schematic representation similar to that of FIG. 9, but with the expandable member collapsed within the third space created by expansion of the expandable member.

FIG. 11 illustrates a schematic representation similar to that of FIG. 10, but with a fixation member of the tissue-separating end effector expanded to anchor the tissue-separating end effector with respect to tissue.

FIG. 12 illustrates a schematic representation similar to that of FIG. 11, but with the expandable member expanded to further separate tissue.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during positioning or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

In accordance with various principles of the present disclosure, a tissue separator includes an expandable member as an end effector configured to separate or dissect tissue. For instance, the expandable member may be used as a blunt instrument configured to separate tissue and/or to perform “third space” procedures and/or procedures in other transluminal spaces of a patient which are generally difficult to access with current devices. It will be appreciated that terms such as medical instrument, tool, device, accessory, etc., may be used interchangeably herein without intent to limit. Typically, a medical instruments which cuts without a sharp cutting edge or application of energy is considered a “blunt” cutting instrument or dissector. In some aspects, a blunt cutting instrument or dissector separates tissue via axial as well as lateral movement with respect to tissue, in contrast with a sharp cutting instrument, such as a knife, electrosurgical knife, blade, scalpel, etc., which typically can cut with axial movement alone). An expandable member formed in accordance with various principles of the present disclosure is inserted through a small incision (e.g., made with a sharper cutting instrument and/or electrosurgical tool) to create and/or to access the “third space”. In some aspects, a fluid (e.g., water, saline, gel, etc.) is injected into target tissue at the treatment site to elevate/lift the tissue (to create a “bleb”) to facilitate cutting of the tissue to create the initial incision, and/or to facilitate insertion of the expandable member (typically in an unexpanded configuration). The expandable member is extended, in an unexpanded configuration, into the tissue (e.g., in the submucosal space or beyond) and then expanded to separate the tissue from within the third space without compromising the integrity of the overlying tissue (e.g., the mucosal and/or submucosal layer). The target tissue is separated by the expandable member almost exclusively by application of sufficient mechanical force to tear connective tissue. Such separation works especially well on boundaries of different tissue types since the mechanical properties of such tissue tend to be different.

When the expandable member is initially inserted into tissue and expanded, the expandable member is expanded to be larger than the size of the initial incision, and therefore is maintained within the third space as it separates tissue. More specifically, although the tissue to be separated may resist advancement and expansion of the expandable member, the expansion of the expandable member to separate the tissue causes the expandable member to be larger than the incision. The expanded expandable member thus cannot rebound through the incision, and the wall in which the incision is formed (and the wall surrounding the incision) resists rebounding of the expandable member.

After initial expansion of the expandable member to create the third space in the treatment site, the expandable member is collapsed, and moved to separate further tissue areas upon further expansion. In accordance with various principles of the present disclosure, if continued separation is desired after initial expansion of the expandable member, an anchoring member is activated to anchor the tissue separator (e.g., prior to further use of the expandable member). As may be appreciated, prior blunt dissectors, such as those using expandable balloons, may rebound upon expansions performed after the initial expansion. Such rebound may be a reaction to expansion against tissue, such as due to the reactionary forces generated by the tissue when the expandable member impacts the tissue. For instance, the initial impact of the expandable member against tissue as the expandable member is expanded and contacts tissue is met by an immediate reactionary force from the tissue. Continued expansion of the expandable member typically results in continued resistance of the tissue to the expansion, creating a constant reactionary force that can push the expandable member back if not adequately counteracted. Moreover, some tissues, such as in the gastrointestinal tract, have elastic properties, initially moving with the expandable member, but then recoiling, exerting a force back onto the expandable member and potentially causing the expandable member to rebound. Once the expandable member has been initially expanded to create the “third space” between tissue layers, the third space provides a path of lesser resistance to the further expansion of the expandable member than a path extending into and separating tissue. If the force exerted by the expandable member to separate further tissue is not sufficient to overcome the resistance of the tissue, or if the tissue is particularly elastic, the expandable member may be pushed back in the direction opposite to its intended expansion direction and towards the already-created third space. This rebounding effect can be particularly problematic in medical procedures where precise positioning and control of the expandable member are crucial. For example, in endoscopic procedures such as endoscopic submucosal dissection (ESD) or per-oral endoscopic myotomy (POEM), the rebounding of the expandable member can cause the expandable member to move away from the target tissue and/or in unintended directions, potentially compromising the effectiveness of the procedure and prolonging the procedure time due to control the rebounding instrument.

In order to address the above-described challenges and shortcomings of the use of expandable members for procedures such as blunt tissue dissection, a tissue separator formed in accordance with various principles of the present disclosure includes a tissue-separating end effector with an expandable member and a fixation member. The fixation member is activated to anchor the tissue-separating end effector of the tissue separator so that expansion of the expandable member is directed into tissue to separate the tissue, with the fixation member resisting the reactionary forces opposing expansion of the expandable member. In some aspects, after the initial expansion of the expandable member to create an initial separation of tissue (e.g., “third space”), the expandable member may be collapsed and the fixation member may be actuated to anchor the tissue separator prior to further actuation of the expandable member. In some aspects, the fixation member is expanded to a fixation size which is larger than a maximum size of expansion of the expandable member so that the fixation member securely fixes the tissue-separating end effector with respect to tissue even when the expandable member is expanded to the maximum size thereof. With the tissue separator thus anchored with respect to the treatment site, the expandable member may be expanded again to further separate tissue. In some aspects, the expandable member and the fixation member may be immediately adjacent to each other when each expanded to a maximum size. In some aspects, the expandable member and the fixation member do not interfere with movement of each other even when one or both are expanded to the maximum size thereof. In some aspects, the cycle of collapsing of the expandable member, actuating of the anchor member, and expanding the expandable member again may be repeated more than once until the desired tissue separation is achieved. In some aspects, the tissue separator is advanced toward tissue to be separated, and the cycle is repeated, to move the tissue separator in an “inchworm” type movement deeper into tissue at the treatment site. Using this sequence, the medical professional can direct and advance the tissue separator into tissue, such as to create submucosal tunnels for example during an E-POEM or G-POEM procedure, or to dissect submucosal tissue during an ESD procedure with minimal or no catheter “back push” or rebound to enhance maneuverability and/or tissue targeting, and/or to be more efficient and effective in separating tissue. Moreover, devices, systems, and method formed in accordance with various principles of the present disclosure allow a medical professional to perform third space endoscopy procedures within the optimal distance from the delivery device (e.g., endoscope), thereby facilitating maintenance of direct visualization of the treatment site during the procedure, and secure directional control of the system and devices, leading to increased safety and efficiency of the procedure.

Various embodiments of tissue separators and associated systems and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

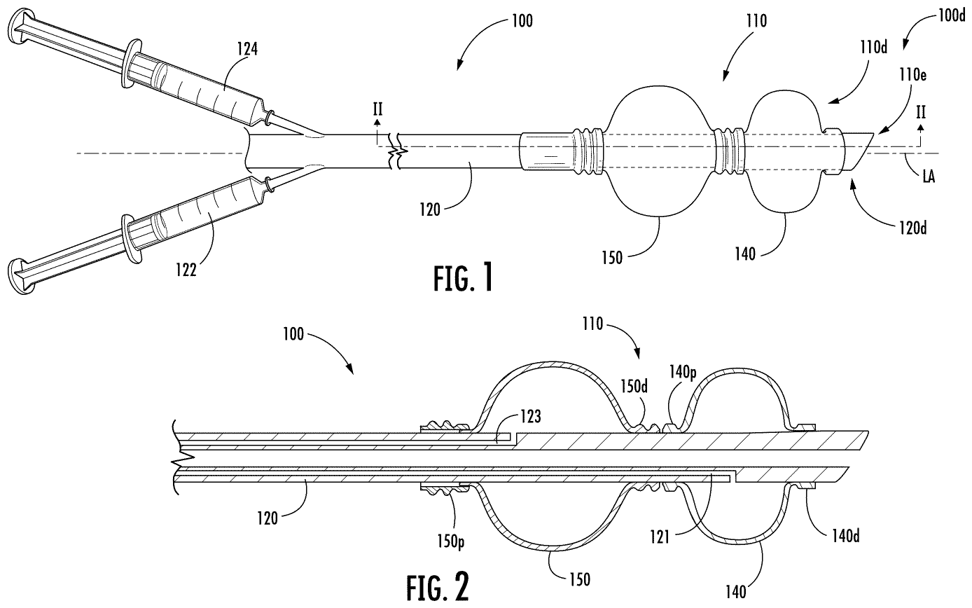

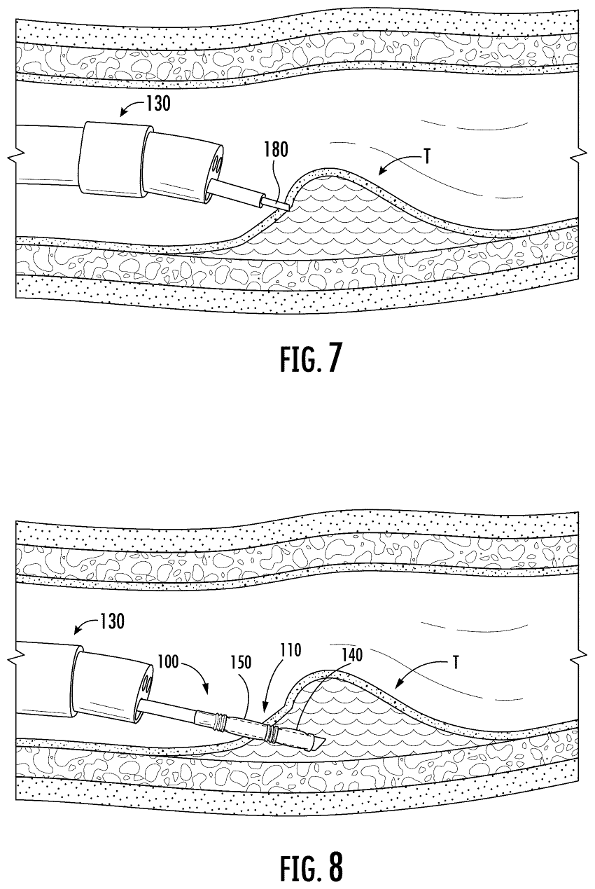

Turning now to the drawings, a tissue separator 100 formed in accordance with various principles of the present disclosure is illustrated in FIG. 1. The tissue separator 100 includes a tissue-separating end effector 110 positioned along a distal end 120d of a flexible elongate member 120. In some aspects, the flexible elongate member 120 is sized, shaped, configured, and/or dimensioned to be navigated within a patient's body to deliver the tissue-separating end effector 110 to a treatment site within the patient's body. For instance, in some aspects, the flexible elongate member 120 is elongated, narrow, and sufficiently flexible to be navigated through curved and even tortuous body passages within a patient. In some aspects, the flexible elongate member 120 is configured to be delivered through a delivery device such as a tubular elongate member 130, such as a sheath or a catheter or an endoscope (such as illustrated in FIG. 7, as known to those of ordinary skill in the art and thus not necessitating full illustration).

In accordance with various principles of the present disclosure, the tissue-separating end effector 110 is sized, shaped, configured, and/or dimensioned to be inserted through an incision created in tissue to perform a third space endoscopic procedure within the potential space inside a tissue wall (e.g., inside the wall of an organ, such as inside the wall of the GI tract, such as in in the submucosal layer of GI tissue). More particularly, the illustrated tissue-separating end effector 110 is sized, shaped, configured, and/or dimensioned to separate tissue, such as to separate the submucosal tissue layer from the muscularis to create and/or to expand the “third space”. The tissue-separating end effector 110 may continue to be used in performing a third space endoscopic procedure. Further in accordance with various principles of the present disclosure, in the example of an embodiment illustrated in FIG. 1, the tissue-separating end effector 110 includes a tissue dissector, such as a blunt tissue dissector, and an anchor engageable with tissue to thereby anchor the tissue-separating end effector 110 against unwanted movements (such as rebounding) during use of the tissue dissector (e.g., upon advancing into tissue to dissect tissue).

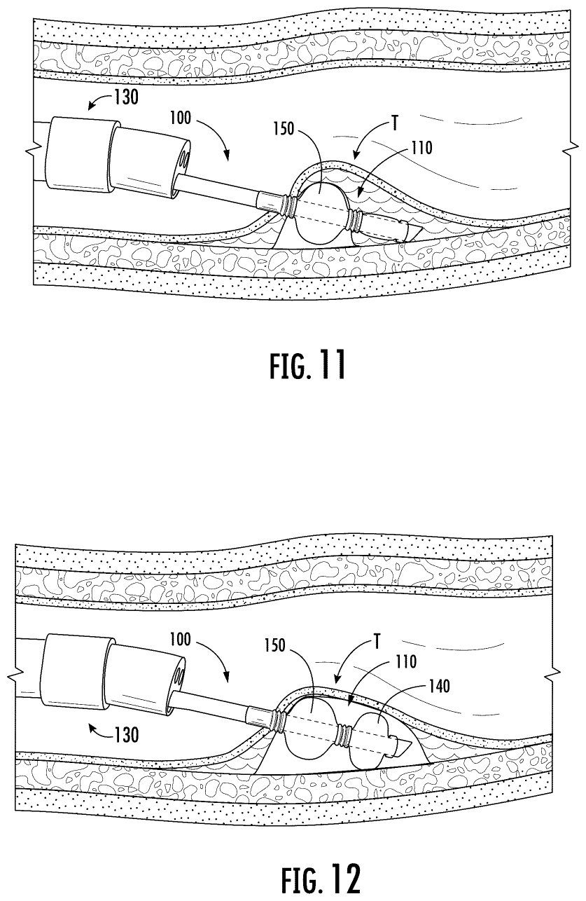

In the example of an embodiment illustrated in FIG. 1, the tissue-separating end effector 110 includes a blunt tissue dissector in the form of an expandable member 140 (which may be considered a dissection member, tissue-separating member, space-creating member, a blunt tissue dissector, etc.). In some aspects, the expandable member 140 is sized, shaped, configured, and/or dimensioned to dissect tissue by separating tissue laterally (in a direction orthogonal to the longitudinal axis LA of the tissue-separating end effector 110) in contrast with cutting into or piercing tissue with a sharp edge (typically achieved by axial movement of a sharp cutting instrument) and/or an energized edge (typically achieved by axial movement of an electrosurgical instrument). For instance, the illustrated example of an embodiment of an expandable member 140 is configured to separate tissue such as upon shifting into an expanded configuration from a collapsed delivery configuration. More particularly, the expandable member 140 is sized, shaped, configured, and/or dimensioned to be inserted into tissue in a collapsed configuration, and to be expanded within the tissue to separate the tissue (e.g., to separate structurally distinct or differentiated layers of the tissue) and to create third space within the tissue upon expansion. For instance, the expandable member 140 is insertable, when in a collapsed configuration (such as illustrated in FIG. 7 and discussed in further detail below), within a tissue wall (inside, and between outer layers of, a tissue wall). Expansion of the expandable member 140 into an expanded configuration within the tissue (such as illustrated in FIG. 11 and discussed in further detail below) creates “third space” within the tissue, such as by separating structurally distinct or differentiated layers of the tissue. The expandable member 140 may be collapsed, moved laterally and/or advanced and/or retracted, and then expanded again, to create a desired or medically indicated tissue separation within the tissue. The collapse, movement, and expansion of the expandable member 140 may be repeated as many times as necessary to create the third space.

In some aspects, the outer dimension (e.g., outer diameter, if generally spherical or cylindrical) of the expandable member 140 in its expanded configuration is at least large enough to separate the submucosal tissue layer from the muscle to create a third space. To further enhance the efficiency of the procedure, the expandable member 140 can be sized (e.g., upon expansion) to increase the initial tissue separation to create a sufficiently large space for a tubular elongate member 130 to enter the third space, such as to deliver further instruments to perform a procedure in the third space. For instance, the outer dimension of the expandable member 140 in its expanded configuration is at least about 8 mm to at least about 13 mm (including increments of 0.1 mm therebetween) to permit entry of an endoscope (typically 8-13 mm in diameter) into the third space created by expansion of the expandable member 140. In some aspects, the outer dimension (e.g., outer diameter, if generally spherical or cylindrical) of the expandable member 140 in its expanded configuration is up to approximately 20 mm. Larger expanded dimensions generally would not be necessary to separate tissue (such as the submucosa from tissue such as the muscle) and create enough space to enter into the third space with the tubular elongate member 130. Also, tissue separation much greater than 20 mm may lead to less control of instruments in the third space, and/or many lead many medical professionals to be nervous about actions being performed too distal to the scope for detailed viewing and fine and/or precise control. In some aspects, the outer dimension includes a radius of curvature at the distalmost end of the expandable member 140 such that the expandable member 140 may be considered by those of ordinary skill in the art to be a blunt dissector. It will be appreciated that the outer dimension of the expandable member 140 in its collapsed configuration may be small enough to be navigated through the tubular elongate member 130, such as through the working channel of a commercially available endoscope such as those familiar to those of ordinary skill in the art.

As discussed above, after the initial expansion of the expandable member 140 to initiate separation of tissue, creating the “third space” in the tissue wall, further expansion, or consequent expansion (after collapsing) of the expandable member 140 within the tissue may be resisted by tissue, and may cause the expandable member 140 to rebound into the already-created third space. In accordance with various principles of the present disclosure, to counter such rebounding, an additional member 150 is provided proximal to the expandable member 140 to fixate/anchor the tissue-separating end effector 110 within the open, dissected space created by the initial expansion of the expandable member 140. The additional member 150 may thus be considered an anchor member, a fixation member, a fixing member, an interlocking member, a locking member, etc., reference being made herein to a fixation member 150 for the sake of convenience and without intent to limit. In some aspects, the fixation member 150 is configured to engage tissue to anchor the fixation member 150, the tissue-separating end effector 110, and optionally the tissue separator 100 in general, with respect to tissue as the expandable member 140 is actuated/operated to separate tissue. In some aspects, the fixation member 150 is expandable to shift from a collapsed configuration, such as for insertion into or moving within the third space, and an expanded configuration in which the fixation member 150 engages surrounding tissue. In some aspects, the outer dimension of the fixation member 150 in its collapsed configuration may be small enough to be navigated through the tubular elongate member 130 with the expandable member 140 (such as described above). Expansion of the fixation member 150 (e.g., once extended out of the tubular elongate member 130) to contact surrounding tissue anchors the tissue-separating end effector 110 with respect to the tissue. In some aspects, the fixation member 150 is larger (e.g., has a slightly larger outer diameter) than the expandable member 140 to create interference between the fixation member 150 and the treatment site without impeding movements of the expandable member 140 to separate tissue. For instance, the expandable member 140 may have a maximum expanded size smaller than a fixation size to which the fixation member 150 expands to anchor the tissue-separating end effector 110 with respect to a treatment site. It will be appreciated that, in some aspects, the relative sizes of the expandable member 140 and the fixation member 150 are determined and/or may be controlled by the amount of fluid within each, and may not be the fully-expanded sizes. In some aspects, the fixation member 150 can have a textured (e.g., with bumps, ridges, etc.) or otherwise modified surface to increase friction between the fixation member 150 and tissue contacted by the fixation member 150. With the fixation member 150 expanded, the expandable member 140 may be expanded to separate tissue, and any counterforce of the tissue in response to repeated expansion of the expandable member 140 is resisted by the fixation member 150. The expandable member 140 may thus continue to separate the tissue without rebounding.

In some aspects, the expandable member 140 is positioned at the distal end 110d of the tissue-separating end effector 110. In some aspects, the expandable member 140 is positioned at the distal end 100d of the tissue separator 100. For instance, the expandable member 140 may be positioned at the distalmost end 110e of the tissue-separating end effector 110 for operation as close to the visualization element of the tubular elongate member 130 (e.g., camera of an endoscope) as possible for improved visualization, identification, and control by the medical professional. In some aspects, the extent of the flexible elongate member 120 distal to the expandable member 140 is reduced or even eliminated to improve directional controllability. The fixation member 150 and the expandable member 140 may be spaced apart from each other by a distance sufficient to allow full expansion of the expandable member 140 (e.g., from an unexpanded configuration) and actuation of the anchor member 150 (e.g., expansion from an unexpanded configuration) without interference from each other. For instance, the distance between the expandable member 140 and the fixation member 150 may be as close as approximately 1 mm, such as if a restriction element (e.g., tied string) separates a single balloon into two chambers to define the expandable member 140 and the fixation member 150. In some aspects, the fixation member 150 is positioned so that little or no space is left between the fully expanded fixation member 150 and the fully expanded expandable member 140. In some aspects, the distal end 140d of the expandable member 140 is mounted adjacent the distal end 120d of the flexible elongate member 120, the proximal end 140p of the expandable member 140 is mounted on the flexible elongate member 120 adjacent, and optionally contacting, the distal end 150d of the fixation member 150, with the proximal end 150p of the fixation member 150 mounted on the flexible elongate member 120 proximal to the location at which the distal end 150d of the fixation member 150 is mounted. In some aspects, there is some interference between the expandable member 140 and the fixation member 150, which does not interfere with relative movement therebetween.

In some aspects, the fixation member 150 and the expandable member 140 are sized, shaped, configured, dimensioned, and/or positioned so that, upon full expansion of both, the only space between the fixation member 150 and the expandable member 140 is to allow lateral movement (e.g., with respect to the longitudinal axis LA of the tissue separator 100) between the fixation member 150 and the expandable member 140. For instance, the expandable member 140 and the fixation member 150 may be sized, shaped, configured, dimensioned, and/or positioned such that upon expansion, the expandable member 140 and the fixation member 150 are no more than about 5 mm to about no more than about 0.5 mm (including any value therebetween) apart from each other. In some aspects, the expandable member 140 and the fixation member 150 are positioned with respect to the tubular elongate member 130 to be operable as close to the tubular elongate member 130 as possible to improve visibility and control thereof. In some aspects, it is desirable not to advance the working tools beyond an optimal viewing distance from an imaging device operably associated with a delivery device such as the tubular elongate member 230. Maintaining close positioning of the expandable member 240 and the fixation member 250 advantageously limits the length of the tissue separator 200 and, in particular, the distance of the expandable member 240 distal to the tubular elongate member 230 (and its optionally associated imaging device).

In some aspects, one or both of the expandable member 140 and the fixation member 150 may be inflatable members. In some aspects, one or both inflatable members may be inflatable balloons. An inflatable member in the form of an inflatable balloon may be made of a compliant (e.g., rubber, silicone, etc.) or a noncompliant (e.g., polyethylene terephthalate (PET), nylon, etc.) or a semi-compliant material (e.g., polyurethane; polyethylene terephthalate (PET), such as at a high enough pressure and/or thin enough wall thickness to allow deformation and compliance of the wall with a given shape; fluorinated ethylene propylene (FEP); and/or other materials, such as with a diameter/pressure compliance curve allowing gradual separation of tissue, opening of a stenosis, etc.), or a combination thereof (e.g., regions of different compliancy, such as a result of different materials, thicknesses, durometers, etc.). For instance, the expandable member 140 may be formed of a compliant material to allow adjustability of the expansion thereof, to achieve the desired degree, magnitude, extent, amount, etc., of tissue separation upon expansion of the expandable member 140. In some aspects, the expandable member 140 may be formed to have a specific shape upon expansion (e.g., a wedge shape), and may optionally be formed from a noncompliant material to expand predictably into such shape. The fixation member 150 may be formed of a noncompliant material to ensure anchoring with respect to surrounding tissue to resist rebounding of the expandable member 140 from tissue being separated. Additionally or alternatively, the fixation member 150 may be formed of a compliant material for increased adjustability and/or control of the expansion of the fixation member 150 (e.g., incremental expansion which nonetheless sufficiently engages tissue) to achieve the desired/necessary engagement with surrounding tissue.

In the example of an embodiment illustrated in FIG. 1 and FIG. 2, the flexible elongate member 120 defines a first lumen 121 having a proximal end in fluid communication with an inflation medium source 122, and a distal end in fluid communication with an expandable member 140 in the form of an inflatable balloon. Optionally, as in the example of an embodiment illustrated in FIG. 1, the fixation member 150 is also in the form of an inflatable balloon, and the flexible elongate member 120 defines a second lumen 123 having a proximal end in fluid communication with an inflation medium source 124, and a distal end in fluid communication with the fixation member 150. It will be appreciated that although the lumens 121, 123 are illustrated in FIG. 2 as defined through the wall of the flexible elongate member 120, one or both of the lumens 121, 123 may be formed as separate elements extending along the flexible elongate member 120. Inflation fluid (air, saline, etc.) from the inflation medium sources 122, 124 is delivered respectively through the lumens 121, 123 to inflate the expandable member 140 and the fixation member 150, respectively. In some aspects, the inflation fluid may be returned to the inflation medium sources 122, 124 to collapse the expandable member 140 and the fixation member 150, respectively. It will be appreciated that although the inflation medium sources 122, 124 are illustrated in FIG. 1 as syringes, they may be any form of a source of an inflation fluid such as known to those of ordinary skill in the art, the present disclosure not being limited in this regard.

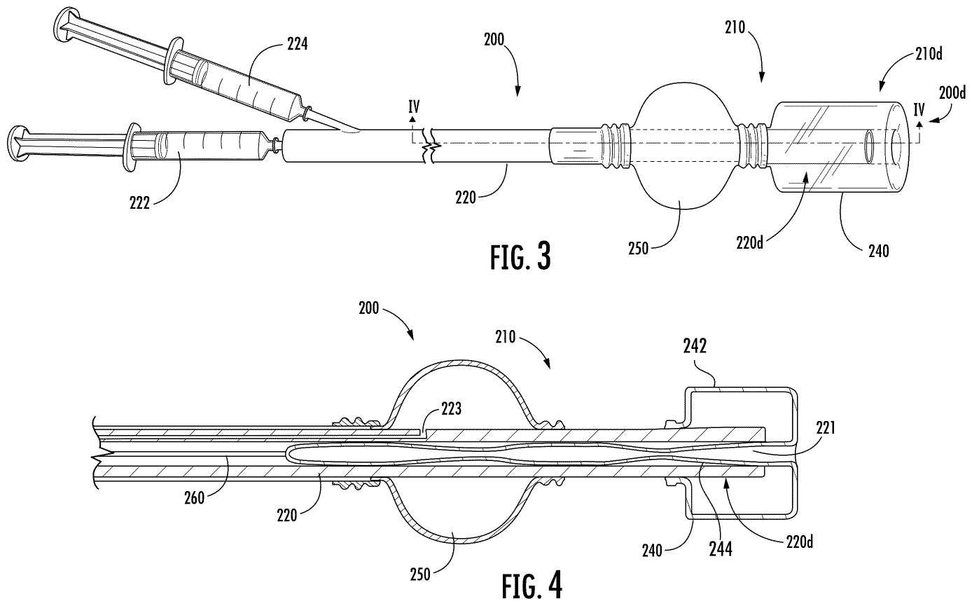

It will be appreciated that one or both of the expandable member 140 and the fixation member 150 may be an expandable member other than an expandable balloon. For instance, in the example of an embodiment of a tissue separator 200 illustrated in FIG. 3 and FIG. 4, a tissue-separating end effector 210 has an expandable member 240 in the form of a tunneling or everting balloon mounted on a distal end 220d of a flexible tubular elongate member 220. A tunnelling or everting balloon typically expands predominantly longitudinally/axially into tissue to separate the tissue, in contrast with expansion in directions orthogonal to the longitudinal axis of the tissue-separating end effector, such as radial expansion, to separate tissue (as in the above-described expandable member 140 in the form of an expandable balloon). For instance, the tissue-separating end effector 210 may be advanced and delivered to a target site with the expandable member 240 inverted and stowed within a lumen 221 defined through the flexible tubular elongate member 220. In some aspects, a periphery 242 (e.g., circumference, boundary, edge, etc.) of the expandable member 240 may be coupled along the exterior/outer surface of the distal end 220d of the flexible tubular elongate member 220, and a radially-inward portion 244 (e.g., a portion along a generally central region, inwardly spaced from the periphery 242) may be invertable into/retractable within the lumen 221 of the flexible tubular elongate member 220. The periphery 242 of the expandable member 240 may be affixed to the distalmost end and/or the exterior of the flexible tubular elongate member 220 in any of a variety of manners, such as mechanical affixing (such as with fixing bands, string, a crimping ring, etc., extending circumferentially around the exterior of the periphery 242 of the expandable member 240 and the exterior of the flexible tubular elongate member 220); and/or bonding such as with adhesive; and/or joining using heat such as by welding, etc., the present disclosure not being limited in this regard. In some aspects, the radially-inward portion 244 of the expandable member 240 is folded, creased, crumpled, or otherwise compacted into the lumen 221 of the flexible tubular elongate member 220. In some aspects, the delivery configuration of a tunnelling or everting balloon must be sufficiently compact to fit within a delivery sheath advanceable through a working channel of an endoscope. As such, the dimensions of an expandable member 240 formed as a tunnelling or everting balloon may be affected by the folding of the balloon into a compact delivery configuration (e.g., for stowing within a lumen of a delivery device). Outer diameters of a tunnelling or everting balloon from about 6 mm to about 11 mm tend to be optimal, although in some embodiments the outer diameter may be as large as about 20 mm with successful operational results.

The expandable member 240 may be caused to expand by filling the lumen 221 of the flexible tubular elongate member 220 with an inflation medium, such as supplied by an inflation medium source 222 (similar to above-described lumen 123 and inflation medium source 122, reference being made to the description thereof, for the sake of brevity, as applicable to the lumen 221 and inflation medium source 222). It will further be appreciated that the inflation medium causes the radially-inward portion 244 of the expandable member 240 to evert out of the lumen 221, such as to be considered the distal end of the expandable member 240, along the distal end 210d of the tissue-separating end effector 210 and/or the distal end 200d of the tissue separator 200, with the periphery 242 being considered the proximal end of the expandable member 240. The inflation medium impacts the inverted portion of the expandable member 240 within the lumen 221 of the flexible tubular elongate member 220 and pushes the expandable member 240 distally out of the lumen 221.

As may be appreciated, the supply of inflation medium may be controlled, as desired, to control inflation or deflation of the expandable member 240, such as medically indicated by the procedure, to control advancement or retraction of the expandable member 240, as may be appreciated by those of ordinary skill in the art. For instance, careful maintenance and/or control of inflation medium pressure, and/or balancing of pressure within the lumen 221 and within the inflation medium source 222, allows for precise control of expansion and retraction of the expandable member 240. The expandable member 240 may thus be extended out of the tubular elongate member 230 with precise control to extend a precise length of the expandable member 240 distally out of the flexible tubular elongate member 220 and into tissue. Selection of material of the expandable member 240 may also influence the operation and control of expansion and retraction of the expandable member 240. For instance, formation of the expandable member 240 from a non-compliant material allows maintained control and a predictable as the expandable member 240 expands out of the flexible tubular elongate member 220, allowing formation of tunnels with consistent diameters within the tissue, and/or precise tissue separation due to defined expansion limits. Optionally, a tether 260 is coupled to the expandable member 240 (e.g., to the radially-inward portion 244 of the expandable member 240), and extends proximally for access by a medical professional for further control of expansion or retraction of the expandable member 240. The tether 260 may be an elongate member, such as, without limitation, a tubular element, a pull wire, a cord, a string, a filament, etc., capable of pulling the expandable member 240 proximally and/or pushing the expandable member 240 distally to control inversion (and storage) or eversion (and extension) of the expandable member 240 with respect to the flexible tubular elongate member 220.

As the expandable member 240 is everted and longitudinally expanded to tunnel into tissue, the eversion of the expandable member 240 may move the wall of the expandable member 240 laterally across tissue to shear apart and separate the tissue. However, expansion of the expandable member 240 outwardly from the flexible tubular elongate member 220 and against tissue to be separated by the tissue-separating end effector 210 may cause the expandable member 240 to impact the tissue without sufficient shear force to separate and to tunnel into the tissue. In prior balloon tunnelling devices, such impact against tissue may cause the expandable balloon member to rebound against the tissue, thus moving the balloon-tunnelling device proximally away from the tissue to be separated, backing the device and system out of and away from the tissue to be separated. In accordance with various principles of the present disclosure, engagement of a fixation member 250 with surrounding tissue inhibits and/or prevents rebounding of the expandable member 240 as the expandable member 240 expands against tissue. The example of an embodiment of a fixation member 250 illustrated in FIG. 4 may be an expandable balloon, supplied with inflation fluid from an inflation medium source 224 via a lumen 223, similar to the inflation medium source and lumen illustrated in and described with reference to FIG. 1 and FIG. 2, reference being made to the above descriptions for the sake of brevity and without intent to limit. It will, however, be appreciated that, as discussed, the fixation member 250 may be in any other appropriate configuration allowing engagement of the fixation member 250 with tissue at the treatment site to inhibit or prevent rebound of the expandable member 240 and the tissue-separating end effector 210 and optionally also the tissue separator 200 in accordance with various principles of the present disclosure.

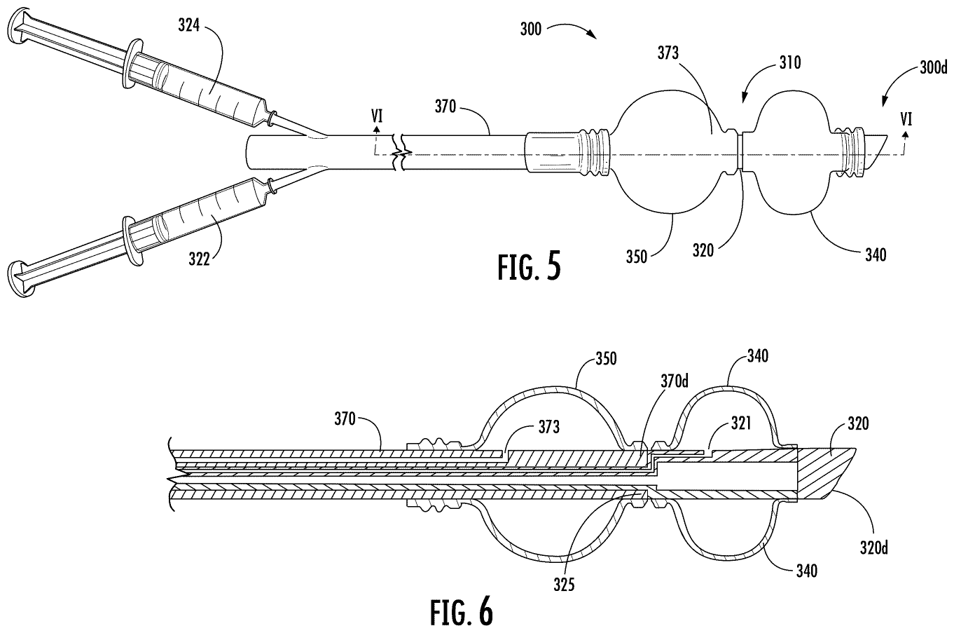

In some aspects, the expandable member and the fixation member of a tissue separator formed in accordance with various principles of the present disclosure are positioned at the distalmost end of a tissue-separating end effector of the tissue separator and as close to the imaging device as possible for optimal visibility and control. In some aspects, it may be desirable to advance the expandable member of a tissue separator formed in accordance with various principles of the present disclosure separately and/or independently from the fixation member. In the example of an embodiment of a tissue separator 300 illustrated in FIG. 5 and FIG. 6, the expandable member 340 of the tissue-separating end effector 310 is provided on the distal end 320d of a flexible elongate member 320, to be positioned at the distal end 300d of the tissue separator 300, and the fixation member 350 is provided on the distal end 370d of a sheath 370 translatably extended over the flexible elongate member 320. For instance, the sheath 370 may define a lumen 371 extending axially therethrough, with the flexible elongate member 320 translatably extendable through the lumen 371. The flexible elongate member 320 may thus be axially advanced with respect to the sheath 370, such as to distally advance the expandable member 340 further into tissue. The flexible elongate member 320 may be axially retracted within the lumen 371 of the sheath 370, such as to move the expandable member 340 closer to the fixation member 350 (e.g., after tissue separation has been performed with the expandable member 340 distally advanced away from the fixation member 350; or if the expandable member 340 is not expanded to its full extent and the medical professional wants to keep the expandable member 340 immediately adjacent the fixation member 350; or in other instances which may occur during performance of a procedure with the tissue separator 300, the present disclosure not being limited in this regard). In some aspects, the expandable member 340 and the fixation member 350 may be expanded or collapsed at different times, sequences, or distances from each other to achieve the desired tissue separation. In some aspects, the expandable member 340 may be “inchwormed” forward (e.g., moved distally incrementally) by retracting the tubular elongate member through which the tissue separator 300 is delivered, while the expandable member 340 is (optionally fully) expanded, to use the system like a reverse “plunger” to separate tissue, such as connected tissue strands. In some aspects, a distal portion of the distal end 320d of the flexible elongate member 320 may define a stop 325 (e.g., shoulder) so that the distal end 320d and the expandable member 340 remain distal to the distal end 370d of the sheath 370. In some aspects, the flexible elongate member 320 and the sheath 370 are delivered together to a treatment site. In some aspects, the flexible elongate member 320 may be advanced towards a treatment site with the fixation member 350 proximal to the treatment site so that the expandable member 340, without the fixation member 350, may be extended through an incision in the treatment site. After initial expansion of the expandable member 340 to create the initial separation of tissue, the fixation member 350 may be advanced distally to be closer to the fixation member 350.

In the example of an embodiment illustrated in FIG. 6, an inflation fluid may be supplied from an inflation medium source 322 and through a lumen 321 defined through the flexible elongate member 320 (on which the expandable member 340 is delivered to a treatment site) to expand the expandable member 340. In some aspects, the inflation fluid may be returned to the inflation medium source 322 to collapse the expandable member 340. Similarly, an inflation fluid may be supplied from an inflation medium source 324 and through a lumen 373 defined through the sheath 370 (on which the fixation member 350 is delivered to a treatment site) to expand the fixation member 350. In some aspects, the inflation fluid may be returned to the inflation medium source 324 to collapse the fixation member 350. It will be appreciated that although the lumens 321, 373 are illustrated as defined through the walls of the flexible elongate member 320 and the sheath 370, respectively, one or both of the lumens 321, 373 may be formed as separate elements extending along the flexible elongate member 320 and/or the sheath 370.

In use, the expandable member and the fixation member of a tissue separator formed in accordance with various principles of the present disclosure may be operated in a sequence such as illustrated in FIGS. 7-12, which may resemble an “inchworm” type movement. As illustrated in FIG. 7, a tubular elongate member 130, such as an endoscope, is advanced to a treatment site T, and a cutting instrument 180 (e.g., a blade, a knife, an electrosurgical knife, a hot knife, etc.) is advanced to the treatment site T to create a small incision. In some aspects, the treatment site T may be elevated or insufflated or otherwise enlarged (such as by injecting a fluid such as saline under the surface/external layer of tissue to create a bleb, such as known to those of ordinary skill in the art) prior to creating the incision. The treatment site T may be a lesion within the wall of a patient's intestines, such as in the submucosal layer of the intestines. The cutting instrument 180 may be withdrawn, and a tissue separator 100 formed in accordance with various principles of the present disclosure may then be advanced to the treatment site T. A tissue-separating end effector 110 of the tissue separator 100 may then be advanced to the treatment site T (e.g., through the tubular elongate member 130) and inserted through the incision and into the boundary between the submucosa and muscle tissue layers, such as illustrated in FIG. 8. In some aspects, the outer diameter of the tissue separator 100, or at least its tissue-separating end effector 110 and/or the flexible elongate member 120 with which the tissue-separating end effector 110 is delivered to the treatment site T, has an outer diameter in the range of approximately 3 mm (e.g., to fit within and be advanced through a 3.2 mm working channel of a typical endoscope). It is noted that FIGS. 7-12 illustrate a tissue separator such as the tissue separator 100 illustrated in FIG. 1 and FIG. 2 merely as an example. It should be appreciated that other tissue separators formed in accordance with various principles of the present disclosure, such as, without limitation, the examples of embodiments illustrated in FIG. 3, FIG. 4, FIG. 5, and/or FIG. 6, may be used instead, without detracting from the described method of separating tissue.

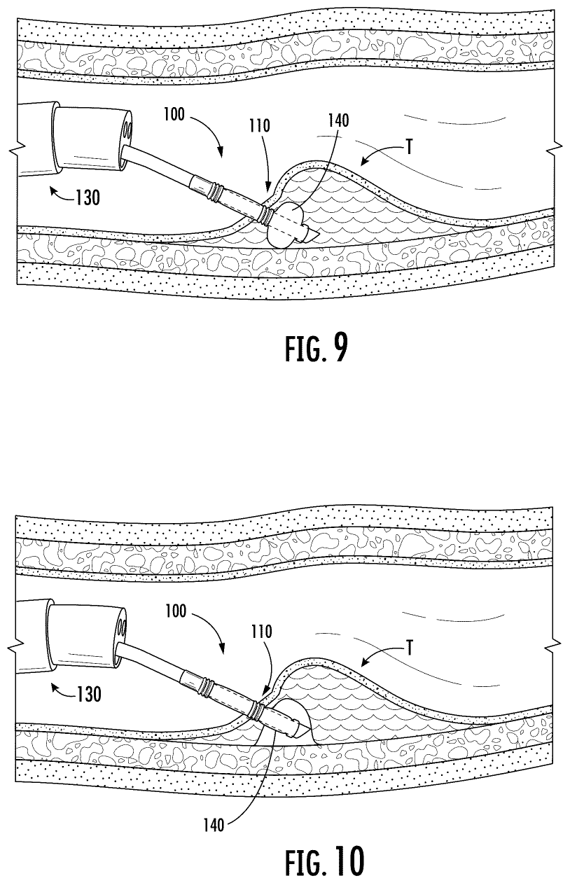

Once the expandable member 140 has been inserted into the treatment site T, the expandable member 140 may be inflated to create an initial separation of tissue layers, such as illustrated in FIG. 9. In some aspects, a marker (e.g., visual marker observable by a medical professional, such as with an imaging device of an endoscope) may be provided along the tissue-separating end effector 110 to assist the medical professional in determining how far to advance the tissue-separating end effector 110 distally into the tissue prior to expanding the expandable member 140. Once tissue at the treatment site T has been separated by the expandable member 140, the expandable member 140 may be collapsed, and the tissue-separating end effector 110 of the tissue separator 100 may be directed and advanced further into the third space created by the tissue-separating end effector 110 and the expandable member 140, such as illustrated in FIG. 10. For instance, the tissue-separating end effector 110, with the expandable member 140 collapsed, may be advanced into tissue surrounding the third space created by the expandable member 140 (such as illustrated in FIG. 9) to initiate further separation of tissue. The fixation member 150 may then be inflated to anchor the tissue-separating end effector 110 (and optionally also the tissue separator 100) with respect to the treatment site T, such as illustrated in FIG. 11. The expandable member 140 may then be expanded once again to separate tissue in the area initially separated by advancement of the tissue-separating end effector 110 with the expandable member 140 in a collapsed configuration, such as illustrated in FIG. 12. The fixation member 150 remains expanded as the expandable member 140 is expanded to separate tissue, as also illustrated in FIG. 12. The tissue-separating end effector 110, within the treatment site T, thereby increases the size of the third space initially created by the initial expansion of the expandable member 140 (as illustrated in FIG. 9). The expandable member 140 may be collapsed, the tissue-separating end effector 110 may be advanced or redirected, and the expandable member 140 expanded again, as many times as necessary to create the desired size, shape, and/or configuration of third space within the treatment site T. It will be appreciated that the fixation member 150 may be collapsed after the expandable member 140 is collapsed to facilitate movement (lateral, proximal, distal, etc., movement) of the tissue-separating end effector 110, such as to target further areas of tissue to separate. The fixation member 150 may then be expanded again to anchor the tissue separator 100 prior to expansion of the expandable member 140 to further separate tissue. This sequential operation (collapse of the expandable member 140 and the fixation member 150, if expanded; expansion of the fixation member 150; and expansion of the expandable member 140) allows for a more controlled and directed tissue separation than achievable by prior tissue separators. The sequential movements of expanding the fixation member 150, expanding the expandable member 140, collapsing the expandable member 140, then collapsing the fixation member 150, then advancing the tissue-separating end effector 110 and repeating the expand/collapse sequence, may resemble an “inchworm” movement (such as noted above). If desired, at any time, or through the entire dissection, the expandable member and the fixation member of the tissue separator may be expanded simultaneously or nearly simultaneously rather than sequentially, the anchoring effect of the fixation member being achieved by the further expansion of the fixation member because of its larger size.

As may be appreciated, a tissue separator formed in accordance with various principles of the present disclosure may be directed and advanced to create third space regions within a patient's anatomy. The use of blunt tissue separators as described herein facilitates tissue separation without the need for application of energy to separate tissue, and thereby reduces the risk of energy-related complications. In some aspects, expansion of an expandable member and a fixation member formed in accordance with various principles of the present disclosure facilitates targeting and progression of tissue separation more effectively and efficiently than prior device which were subject to rebounding upon application of a tissue-separating force to tissue. In some aspects, the use of a blunt separator, such as an expandable member as described herein, with an interlocking/anchoring feature, such as a fixation member as described herein, provides improved steerability and control of blunt dissection than achievable with prior blunt dissectors. In some aspects, a lumen may be provided through the flexible elongate member with which the expandable member of the tissue separator is delivered, and another device, such as cauterization tools, may be passed through such lumen if needed. In some aspects, a tissue separator formed in accordance with various principles of the present disclosure may be advanced in multiple directions to create multiple tunnels (e.g., submucosal tunnels), which may then be connected, such as by cutting the walls between the tunnels, to create an open third space within the tissue in which the tunnels were formed. Various other procedures which may be performed advantageously with a tissue separator formed in accordance with various principles of the present disclosure include, but are not limited to, per oral endoscopic myotomy (POEM), endoscopic submucosal dissection (ESD), Zenker's diverticulotomy (ZPOEM), endoscopic gastric pyloromyotomy (GPOEM), submucosal tunneling endoscopic resection (STER), and tunneling recanalization of the esophagus (POETRE), among others. For instance, tunnels may be advantageously created during an E-POEM or G-POEM procedure, or to dissect submucosal tissue during ESD with minimal or no device “back-push”. By fixing the tissue separator in place with respect to tissue to inhibit, and preferably to prevent, back out, a medical professional need not apply constant forward pressure on the tissue separator to overcome rebound forces of the tissue, allowing for easier stabilization and maneuvering of the system. A tissue-separating end effector formed in accordance with various principles of the present disclosure thus enhances maneuverability, tissue targeting, and efficiency of a tissue separator device and system, and allows for safer, more efficient, more controllable tissue separation procedures, potentially reducing procedure times and improving outcomes for patients.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like. For instance, principles of the present disclosure may be applied to various expandable members used within a patient, such as within tissue. Principles of the present disclosure may be used to solve the ongoing problem of catheter push-back upon re-inflation of an inflatable member within tissue. It will further be appreciated that delivery devices are not limited to a catheter or endoscope, and may include a variety of medical devices for accessing body passageways, including, for example, duodenoscopes, catheters, ureteroscopes, bronchoscopes, colonoscopes, arthroscopes, cystoscopes, hysteroscopes, and the like.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects, features, components, and structures of a tissue separator and use thereof, such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A tissue separator capable of being navigated transluminally to a treatment site within a patient and insertable through an incision formed in tissue at the treatment site to create a third space within the tissue, said tissue separator comprising:
a flexible elongate member configured to be navigated within a curved or tortuous body passage to the treatment site and having a distal end; and
an end effector positioned along the distal end of said flexible elongate member and configured to be inserted through the incision in the tissue at the treatment site, said end effector comprising an expandable member at the distal end of said flexible elongate member, and a fixation member adjacent and proximal to said expandable member.

2. The tissue separator of claim 1, wherein said expandable member is an inflatable balloon.

3. The system of claim 2, wherein said fixation member is an inflatable ballon, and said flexible elongate member defines a first lumen fluidly communicating inflation medium to said expandable member, and a second lumen fluidly communicating inflation medium to said fixation member.

4. The system of claim 1, wherein said expandable member is a tunneling balloon.

5. The system of claim 1, wherein said fixation member is expandable to a fixation size, and said expandable member is expandable to a maximum size smaller than the fixation size of said fixation member.

6. The system of claim 5, wherein when said fixation member is expanded to the fixation size and said expandable member is expanded to the maximum size thereof, said expandable member and said fixation member are immediately adjacent each other without interfering with movement of each other.

7. The system of claim 1, wherein said fixation member is an inflatable balloon.

8. The system of claim 1, wherein said fixation member is mounted on a distal end of a sheath axially translatable over said flexible elongate member.

9. A system for separating tissue within a patient to create a third space within the tissue, said system comprising:

10. a cutting device capable of being navigated transluminally within a patient's body to form an incision in the tissue; and

11. a tissue separator capable of being navigated transluminally to the treatment site and insertable through an incision formed by said cutting device and into the tissue, said tissue separator comprising:

12. a flexible elongate member configured to be navigated within curved or tortuous body passages to the treatment site and having a distal end;

13. an end effector positioned along the distal end of said flexible elongate member and configured to be inserted through the incision formed by said cutting device, said end effector comprising an expandable member at the distal end of said flexible elongate member, and a fixation member proximal to said expandable member.

14. The system of claim 9, wherein said expandable member is an inflatable balloon.

15. The system of claim 9, wherein said expandable member is a tunneling balloon.

16. The system of claim 9, wherein said fixation member is expandable to a fixation size, and said expandable member is expandable to a maximum size smaller than the fixation size of said fixation member.

17. The system of claim 12, wherein when said fixation member is expanded to the fixation size and said expandable member is expanded to the maximum size thereof, said expandable member and said fixation member are immediately adjacent each other without interfering with movement of each other.

18. The system of claim 9, wherein said fixation member is mounted on a distal end of a sheath axially translatable over said flexible elongate member.

19. The system of claim 9, wherein said fixation member is an inflatable balloon.

20. A method of creating third space within tissue within a patient's body, said method comprising:

21. inserting a distal end of an end effector of a tissue separator through an incision in tissue within a patient's body and into the tissue;

22. expanding an expandable member on the distal end of the end effector within the tissue to separate the tissue and to create an initial third space within the tissue;

23. collapsing the expandable member;

24. expanding a fixation member of the end effector within the initial space within the tissue to anchor the end effector with respect to the tissue; and

25. expanding the expandable member to further separate tissue to expand the size of the initial third space within the tissue.

26. The method of claim 16, further comprising collapsing the expandable member, collapsing the fixation member, advancing the end effector within the tissue, expanding the fixation member, and expanding the expandable member to increase the size of the third space within the tissue.

27. The method of claim 16, wherein the expandable member is an inflatable balloon, and expanding the expandable member comprises inflating the expandable member.

28. The method of claim 18, wherein the fixation member is an inflatable balloon, and expanding the fixation member comprises inflating the fixation member.

29. The method of claim 16, wherein expanding the expandable member to further separate tissue comprises expanding the expandable member up to a size at which the expandable member is immediately adjacent the fixation member.
